(19) United States
(12) Patent Application Publication
(10) Pub. No.: US 2020/0179607 A1
(43) Pub. Date: Jun. 11, 2020

(54) INJECTION DEVICE WITH PRIMING ARRANGEMENT

(71) Applicant: Eli Lilly and Company, Indianapolis, IN (US)

(72) Inventor: Jared Alden Judson, Medford, MA (US)

(21) Appl. No.: 16/607,903

(22) PCT Filed: Apr. 25, 2018
(86) PCT No.: PCT/US2018/029316

§ 371 Date: Oct. 24, 2019

Related U.S. Application Data
(60) Provisional application No. 62/500,004, filed on May 2, 2017.

(51) Int. Cl. A61M 5/24; A61M 5/31; A61M 5/315

(52) U.S. Cl. CPC A61M 5/2422; A61M 5/3146; A61M 2005/2488; A61M 5/31511; A61M 5/3158

(57) ABSTRACT

An injection device for dispensing medication from a medicament cartridge is disclosed having a needle assembly at one end and a piston disposed opposite the needle assembly wherein advancement of the piston within the cartridge discharges medication from the needle assembly. The injection device may include a housing assembly and a driving assembly adapted to drive the piston and thereby discharge medication from the needle assembly. A cartridge retainer may be securable to the housing assembly and adapted to retain the cartridge. Securing the cartridge retainer to the housing assembly with a retained cartridge may move the piston of the cartridge into contact with the driving assembly and perform a priming procedure. The cartridge retainer may be securable to the housing assembly by rotationally engaging a helical thread with a helical groove. A ratcheting mechanism may be used to prevent the rotational disengagement of the cartridge retainer from the housing assembly.

### CROSS REFERENCE TO RELATED APPLICATION

This application claims priority to U.S. Provisional Patent Application Ser. No. 62/500,004, filed May 2, 2017, the disclosure of which is hereby expressly incorporated by reference herein in its entirety.

### BACKGROUND

The present disclosure relates to medical delivery devices such as injection devices.

Conventional injection devices are often used to inject a medicament into patient. Such devices commonly employ a plunger or similar elongate rod to advance a piston to dispense the medicament. Such devices might be refillable wherein retraction of the piston is used to draw-in the medicament before the injection process, or, the device might be configured to be used with disposable containers that have been prefilled with the medicament.

For example, injection pens that receive disposable cartridges containing insulin are often used by diabetes patients. Such pens generally include an elongate rod that acts on a piston within the cartridge. As the rod advances the piston, the medicament within the cartridge is dispensed through a needle and into the patient.

Various other configurations are also known. For example, Judson, WO 2016/149014 A1, published Sep. 22, 2016, and U.S. provisional patent application Ser. No. 62/134,865 filed Mar. 18, 2015, the disclosures of both of which are incorporated herein by reference, disclose a dispensing device with a drive mechanism having converging ramps. The disclosed device includes a plunger having two separate plunger arms that are pivotally connected together at the button end that is pushed by the user to eject medicament.

When using such injection devices, it is often desirable to prime the cartridge containing the medication before the initial injection. Such priming takes place after attaching a needle assembly to the cartridge and involves advancing the piston of the cartridge a small amount to cause one or more droplets of medication to be discharged from the needle assembly. This priming process forces out any air contained in the reservoir of the cartridge and the air within the passage of the needle assembly and fills such spaces with the medication. This priming procedure improves the accuracy of the following injection process because the advancement of the piston during the injection process will discharge only medication from the needle assembly and will not require that the initial advancement of the piston discharge air.

For cartridges which are configured for only a single use, there will not be a need for any subsequent priming of the cartridge after the initial priming of the cartridge. For multi-use cartridges, the initial priming of the cartridge is often sufficient to provide the necessary accuracy for the initial injection and any subsequent injections. In some situations employing multi-use cartridges, however, it may be desirable to prime the cartridge before each use.

The priming of cartridges containing medication is widely practiced and several different methods can be employed. One simple procedure is for the user of the device to manually advance the drive mechanism and cartridge piston to cause a small quantity of medication to be ejected to thereby prime the cartridge.

### SUMMARY

The present disclosure provides a convenient and user-friendly injection device and method for priming a medicament cartridge.

In an illustrated embodiment, the action used to prime the cartridge is different from the action used to discharge the medication during an injection procedure.

In an illustrated embodiment, prior to the initial use of the cartridge, the act of installing the cartridge in the injection device simultaneously primes the cartridge.

### BRIEF DESCRIPTION OF THE DRAWINGS

The above mentioned and other features of this disclosure, and the manner of attaining them, will become more apparent and will be better understood by reference to the following description of embodiments of the invention taken in conjunction with the accompanying drawings, wherein:

FIG. 1 is an exploded view of an injection device.

FIG. 2 is a top view of the injection device with the plunger in the advanced position with a housing member removed.

FIG. 3 is a top view of the injection device with the plunger in the retracted position with a housing member removed.

FIG. 4 is a top view of the injection device with the plunger in a partially advanced position with a housing member removed.

FIG. 5 is a top view of a housing member.

FIG. 5A is a perspective view of the housing member of FIG. 5

FIG. 5B is an end view of the housing member of FIG. 5.

FIG. 5C is an end view of the housing member of FIG. 5.

FIG. 5D is a side view of the housing member of FIG. 5.

FIG. 5E is a perspective view of the housing member of FIG. 5.

FIG. 6 is a top view of another housing member.

FIG. 6A is a perspective view of the housing member of FIG. 6.

FIG. 6B is an end view of the housing member of FIG. 6.

FIG. 6C is an end view of the housing member of FIG. 6.

FIG. 6D is a side view of the housing member of FIG. 6.

FIG. 6E is a perspective view of the housing member of FIG. 6.

FIG. 7 is a perspective view of a cartridge holder.

FIG. 7A is a side view of the cartridge holder.

FIG. 7B is another side view of the cartridge holder.

FIG. 7C is a cross sectional view taken along line 7C-7C of FIG. 7B.

FIG. 7D is a cross sectional view taken along line 7D-7D of FIG. 7B.

FIG. 7E is an end view of the cartridge holder.

FIG. 7F is another end view of the cartridge holder.

FIG. 8 is a perspective view of the plunger.

FIG. 8A is perspective view of the plunger.

FIG. 8B is an end view of the plunger.

FIG. 8C is a side view of the plunger.

FIG. 8D is a side view of the plunger.

FIG. 8E is a top view of the plunger.

FIG. 9 is a top view of the injection device before the initial priming of the cartridge.

FIG. 9A is a cross sectional view taken along line 9A-9A of FIG. 9.

FIG. 9B is a cross sectional view taken along line 9B-9B of FIG. 9.

FIG. 9C is a cross sectional view taken along line 9C-9C of FIG. 9.

FIG. 10 is a top view of the injection device midway through the initial priming of the cartridge.

FIG. 10A is a cross sectional view taken along line 10A-10A of FIG. 10.

FIG. 10B is a cross sectional view taken along line 10B-10B of FIG. 10.

FIG. 10C is a cross sectional view taken along line 10C-10C of FIG. 10.

FIG. 11 is a top view of the injection device after completing the initial priming of the cartridge.

FIG. 11A is a cross sectional view taken along line 11A-11A of FIG. 11.

FIG. 11B is a cross sectional view taken along line 11B-11B of FIG. 11.

FIG. 11C is a cross sectional view taken along line 11C-11C of FIG. 11.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

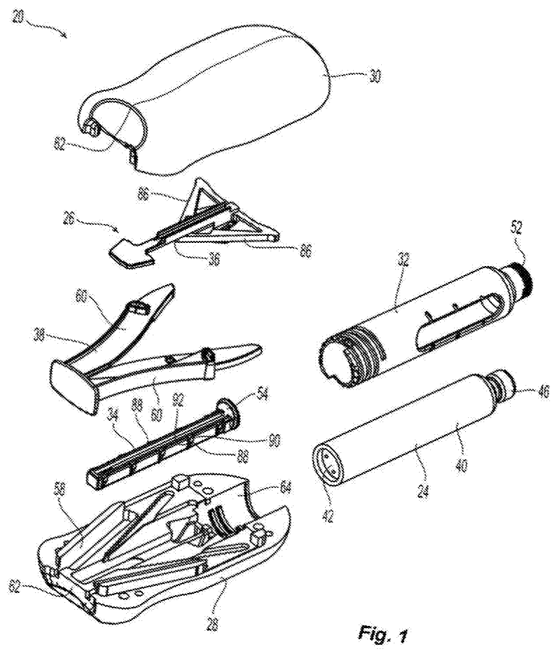

An exemplary medication dispensing device 20 is shown in FIG. 1. Device 20 includes a housing assembly 22 which secures a medicament cartridge 24 to the device, and a drive assembly 26. The housing assembly 22 includes first and second housing members 28, 30 and a cartridge holder 32 which is also referred to herein as a cartridge retainer. The driving assembly 26 includes a drive stem 34, a driver 36 and a plunger 38.

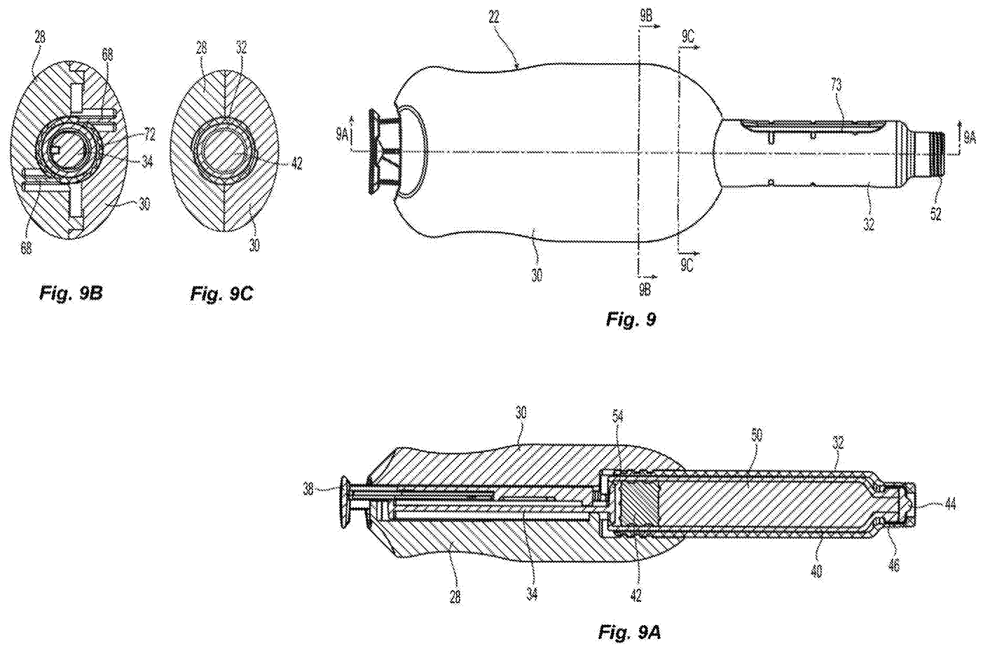

As best seen in FIG. 9A, cartridge 24 is a conventional medicament cartridge including a barrel 40 with an interior reservoir sealed at one end by a slidable piston 42 and sealed at the other end by a septum 44 held by a crimp ring 46. A needle assembly 48 is mounted to an externally threaded end 52 of cartridge retainer 32 pierces the septum 44 to provide an outlet during dispensing of the medication 50 filling the barrel reservoir. A removable cap (not shown) can be used to cover the needle assembly when device 20 is not in use. Cartridge 24 may hold multiple doses of medicine, or a single dose, depending on the design of device 20. The shown device 20 is a multiple use, fixed dose device, meaning that the device may be used to deliver the same dose on multiple occasions from a single cartridge 24. The shown cartridge is merely one form of container from which the drive assembly 26 may force an emptying thereof, as, for example, a container that is compressed by action of the drive assembly alternatively may be employed.

Drive stem 34 of drive assembly 26 projects into barrel 40 and has a foot 54 that engages piston 42. As drive stem 34 is advanced along drive axis 56, foot 54 advances piston 42 within barrel 40 to discharge medication 50 from needle assembly 48. The advancement of plunger 38 biases driver 36 forward along axis 56 to thereby advance drive stem 34 into engagement with piston 42 when the device is being used to discharge medication.

Driver 36 directly engages drive stem 34 and is constrained by interior surfaces of housing assembly 22 to be axially translatable and rotatably fixed therein. The interaction of converging ramp surfaces 58 located on the interior surfaces of housing assembly 22 and plunger arms 60 provides a mechanical advantage allowing the user to depress the plunger and discharge the medicament using less force than would otherwise be required if the device did not provide a mechanical advantage.

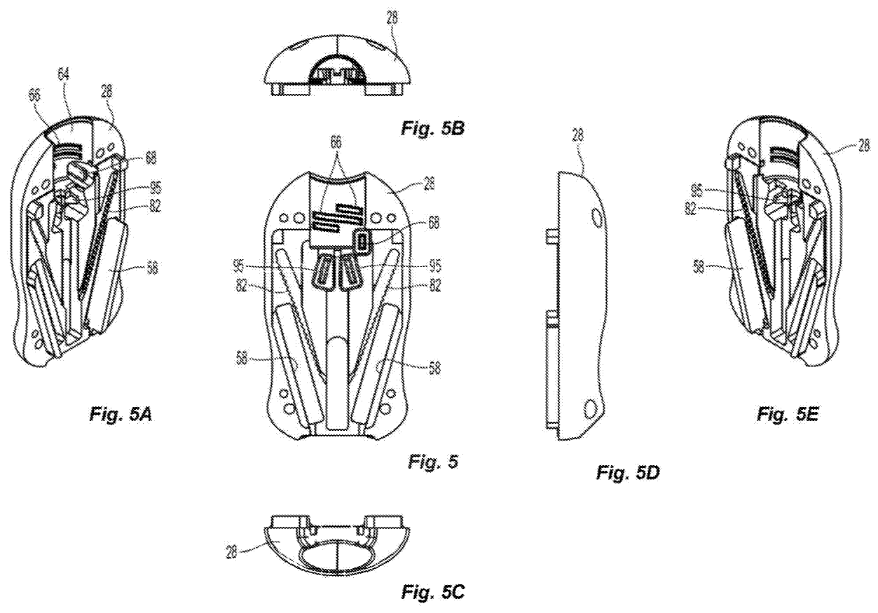

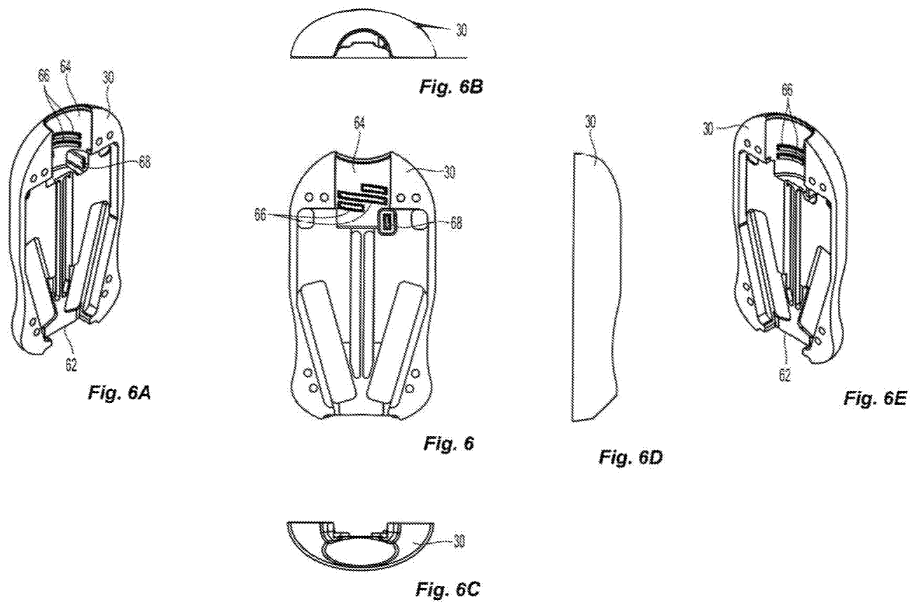

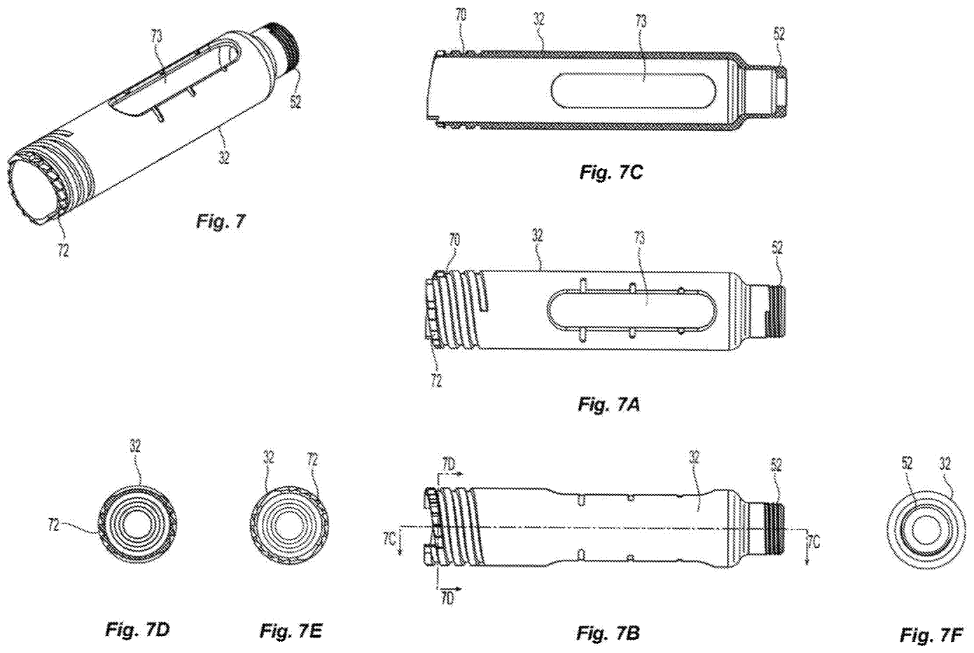

Housing members 28, 30 are shown in greater detail in FIGS. 5, 5A-5E and FIGS. 6, 6A-6E. The housing members 28, 30 are secured together and capture driver 36 and plunger 38 therebetween while still permitting relative movement of these components. The housing members 28, 30 can be secured together with adhesive, threaded fasteners, snap-fit connections or other suitable means.

Housing members 28, 30 have a cylindrical opening 64 at one end which includes thread members 66. Each of the illustrated housing members also includes a ratchet member 68 located in or adjacent the cylindrical opening 64. Cartridge retainer 32 includes helical grooves 70 and ratchet teeth 72 on the end which engages housing members 28, 30. Grooves 70 define double-start helical grooves which receive thread members 66. Ratchet teeth 72 are located on spiral lands between grooves 70. Interaction between ratchet members 68 and ratchet teeth 72 allow cartridge retainer 32 to be rotated inwardly into the cylindrical opening 64 but prevent retainer 32 from being rotated back out. Alternative embodiments could employ ratchet teeth on the housing assembly and a ratchet member on the cartridge retainer.

To detach cartridge retainer 32, and the cartridge 24 located therein, from housing members 28, 30, housing members 28, 30 must be separated. Device 20 may be a single use dispenser intended to be disposed of after emptying the initial cartridge 24. For such single use dispensers, housing members 28, 30 may be permanently secured together. Alternatively, threaded fasteners or other suitable attachment means may be employed to allow housing members 28, 30 to be detached from each other and re-secured by the user to thereby allow the user to replace an empty cartridge 24 with a full cartridge.

Cartridge retainer 32 and housing members 28, 30 are configured to facilitate the initial priming of cartridge 24. For example, device 20 may be shipped with a full cartridge 24 located within retainer 32 and with retainer 32 not fully rotated into its final position. Before the first injection, the user mounts needle assembly 48, and then rotate cartridge retainer 32 until thread members 66 hit the end of helical grooves 70 which blocks or prevents the further rotation of retainer 32. As retainer 32 is twisted into its installed position, it moves cartridge 24 toward foot 54 of drive stem 34. Drive stem 34 may be positioned so that this twisting of cartridge retainer 32 into its installed position biases piston 42 forward a small amount to thereby prime cartridge 24 for its first dispensing of medicine 50. When needle assembly 48 is mounted, it pierces septum 44 and is ready to dispel air located in needle assembly 48 and dispense a small amount of medicine before cartridge retainer 32 is twisted into its final position to thereby allow the securement of cartridge retainer 32 to prime cartridge 24.

Window 73 in cartridge retainer 32 allows a user to view cartridge 24 and thus, visually determine the quantity of medicine remaining in cartridge 24 provided that barrel 40 is transparent or at least partially transparent. Window 73 may also be positioned such that when cartridge 32 is disposed in its installed position it assumes a predefined position relative to housing assembly 22 to thereby provide the user with confirmation that cartridge retainer 32 is in its proper installed position.

The priming of cartridge 24 by the rotation of cartridge retainer 32 relative to housing members 28, 30 is discussed in greater detail below.

Plunger 38 projects from housing assembly 22 opposite cartridge retainer 32 whereby the user can retract plunger 38 by pulling it outwardly from housing assembly 22 to place device 20 in condition for an injection procedure and push plunger 38 into housing assembly 22 to cause the injection procedure. Each of the housing members 28, 30 define a portion of the opening 62 through which plunger 38 projects. The housing member 28 also defines ramp surfaces 58. Ramp surfaces 58 constrain the outward movement of plunger arms 60 as plunger 38 is advanced toward needle assembly 48.

Each of the plunger arms 60 include a slide member 74. Each of the slide members 74 slide along one of the ramp surfaces 58 as plunger 38 is advanced within housing assembly 22. The inner portions of plunger arms 60 includes a recessed area 76 that allows the recessed areas 76 of both arms to overlap as arms 60 are biased toward each other as plunger 38 is pulled out of housing assembly 22 in a manner similar to the way the blades of a pair of scissors overlap when the scissors are closed. Detents 78 are located in recessed areas 76 of each plunger arm. Detents 78 engage each other when plunger 38 is retracted from housing assembly 22 in preparation for an injection to thereby hold plunger 38 in the retracted position. When the user begins pushing plunger 38 into housing assembly 22 to initiate an injection, the force applied by the user will first disengage detents and then begin advancing plunger 38. The force required to overcome the attachment of detents 78 is relatively light as further discussed below.

As plunger 38 is advanced, an engagement surface 84 proximate the forward tip of plunger arms 60 engages inclined surfaces 86 of driver 36. The gap between ramp surfaces 58 and inclined surfaces 86 is insufficient to allow plunger arms 60 to pass therebetween, thus, as slide member 74 advances along ramp surface 58 engagement surfaces 84 on arms 60 will exert a force on inclined surfaces 86 and push driver 36 forward toward needle assembly 48. In this regard it is noted that inclined surfaces 86 and ramp surfaces 58 are not parallel and thus engagement surfaces 84 slide along inclined surfaces 86 until they begin to push driver 36 forward and as the plunger arms 60 advance, engagement surfaces 84 both slide along and push inclined surfaces 86.

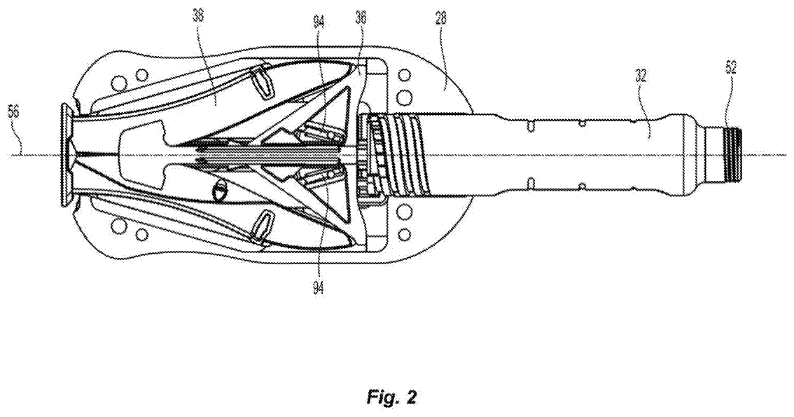

Drive stem 34 includes a plurality of ratchet teeth 88 encircling the central stem of drive stem 34. The surface 90 of ratchet teeth 88 facing needle assembly 48 are inclined while the opposite side of teeth 88 define a stop surface 92 extending perpendicular to axis 56. Driver 36 includes two pawl members 94 that engage drive stem 34 and can be seen in FIG. 2. As driver 36 is advanced by plunger arms 60, pawl members 94 engage one of the stop surfaces 92 on drive stem 34 and push drive stem 34 forward whereby foot 54 pushes piston 42 forward and causes medication 50 to be discharged through needle assembly 48.

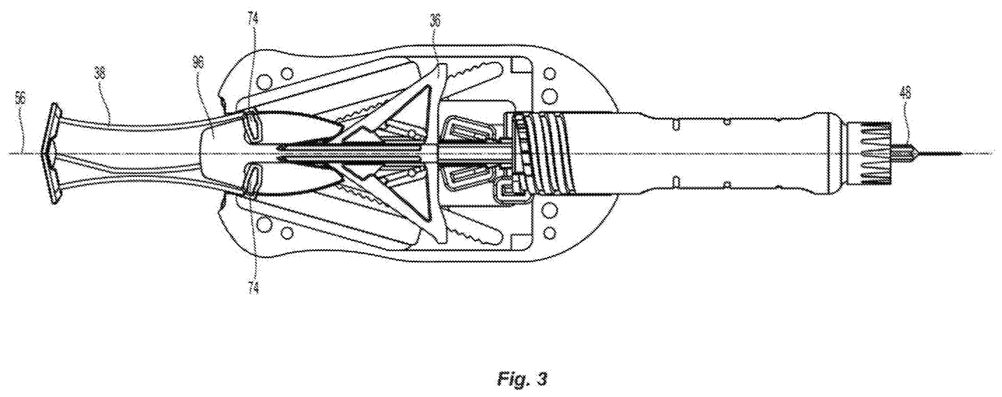

When plunger 38 is retracted, slide members 74 engage retraction head 96 and pull driver 36 rearwardly during the final phase of the retraction (see FIG. 3). As driver 36 is retracted, pawls 94 slide along drive stem 34. When pawls 94 encounter a ratchet tooth 88, the inclined surface 90 of the ratchet tooth allows pawls 94 to slide over the ratchet tooth 88.

Housing member 28 includes two ratchet members 95 (FIG. 5) that engage drive stem 34 and prevent the retraction of drive stem 34 when plunger 38 is being retracted. Ratchet members 95 have a sloped surface that allows drive stem 34 to be advanced toward needle assembly 48 as can be seen in FIGS. 5A and 5E.

Each of the plunger arms 60 also includes a braking member 80 and housing member 28 includes a pair of braking tracks 82. As slide members 74 travel along ramps 58 braking members 80 will remain spaced apart from braking track 82 in both the forward and rearward directions so long as the force applied by the user to button end 110 of plunger 38 during advancement of the plunger is not excessive. Braking tracks 82 are offset from the paths of braking members 80 when slide members 74 travel along ramps 58 and both tracks 82 and ramps 58 are disposed on the same housing member 28 in the illustrated embodiment. When the user applies an excessive force to button end 110 of plunger 38 when advancing plunger 38 into housing assembly 22, the excessive force will bias braking members 80 into braking track 82 to thereby increase the resistance to the force being applied by the user. Illustrated braking member 80 and braking track 82 include undulations or teeth that will engage and disengage as braking member 80 advances along braking track 82 causing an audible chatter and also causing the user to feel a resistance force that include repetitive increases or spikes in resistance as the individual teeth of braking member 80 and braking track 82 engage and then release.

Braking member 80 includes a plurality of teeth 98 that face braking track 82. Braking track 82 includes a plurality of teeth 100. When braking member 80 engages track 82, the teeth 98 engage teeth 100 and slide along track 82 with a series of impacts as teeth 98 impact teeth 100 and then release and engage the next set of teeth 100 on track 82.

As plunger arm 60 is pushed forward with braking member 80 in engagement with braking track 82, this engagement will increase the resistance felt by the user and also create “chatter” as the teeth on braking member 80 pass over the teeth 100 on braking track 82. This chatter may be felt by the user and creates an audible sound that may be heard by the user. The increased resistance of forward movement of plunger 38 and the chatter caused by the series of impactful engagements of teeth 98 with teeth 100 will alert the user that they are pressing on plunger 38 in an excessively forcible manner and that they should lessen the force being applied to plunger 38.

The use of a braking mechanism with device 20 is also discussed in U.S. provisional application Ser. No. 62/500,055, filed May 2, 2017 and entitled INJECTION DEVICE WITH PLUNGER ARRANGEMENT AND CONVERGING RAMPS filed by the same Applicant as the present application, the disclosure of which is incorporated herein by reference. Various other device configurations, which do not necessarily include a braking function, may alternatively be employed with the priming feature of the present application. For example, WO 2016/149014 A1 discloses one example of such an alternative device. Furthermore, other injection device designs, such as those which do not employ a plunger having arms that engage inclined ramps to drive the plunger, may also be used with the priming arrangement of the present application.

In the illustrated example, arms 60 of plunger 38 are part of the same integral part and are connected together via material bridge 108. Material bridge 108 forms the central portion of button end 110 of plunger 38 and connects the two opposing halves 112 of button end 110.

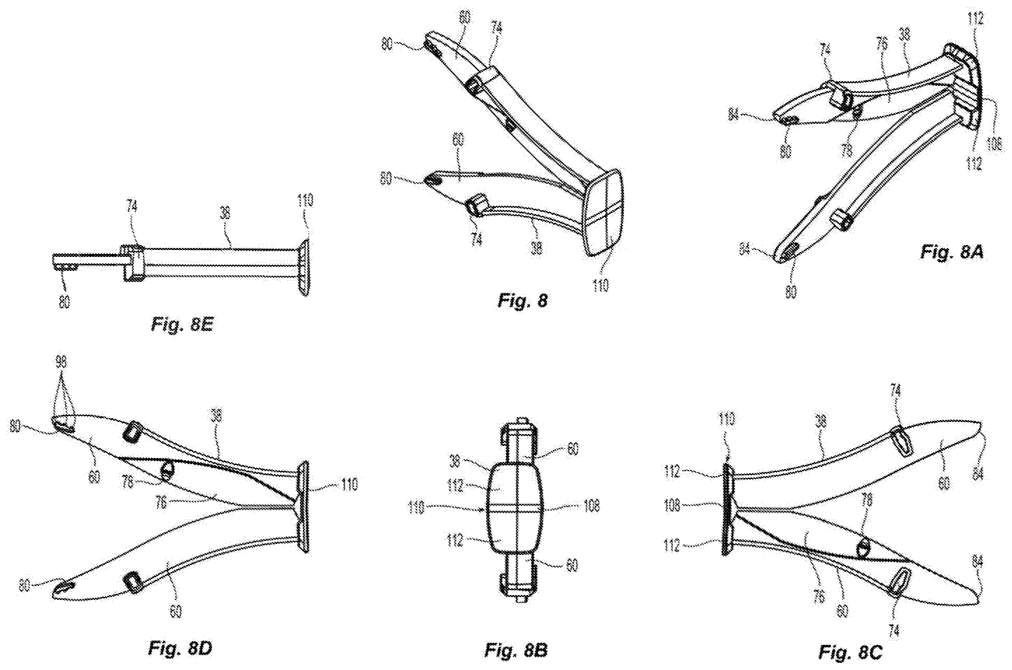

In one embodiment, material bridge 108 is thinner than the two opposing halves 112 of button end 110 and is sufficiently thin to flex as plunger 38 is moved along axis 56. Plunger 38 is a single molded part and the original molded configuration of plunger 38 is the configuration shown in FIGS. 8C and 8D. In this configuration, opposing arms 60 are spread apart. Arms 60 are slightly biased together when plunger 38 is assembled into housing assembly 22 in the fully advanced position. FIGS. 2, 2A-2C show plunger 38 in the fully advanced position. As plunger 38 is retracted outwardly from housing assembly 22, arms 60 are biased toward each other. Material bridge 108 flexes as arms 60 move toward and away from each other. In the illustrated embodiment, however, the stress applied to material bridge 108 during the range of movement resulting from normal operation of device 20 does not reach the yield stress of the material forming material bridge 108. As a result, material bridge 108 exerts a biasing force that seeks to place arms 60 in the configuration shown in FIG. 2.

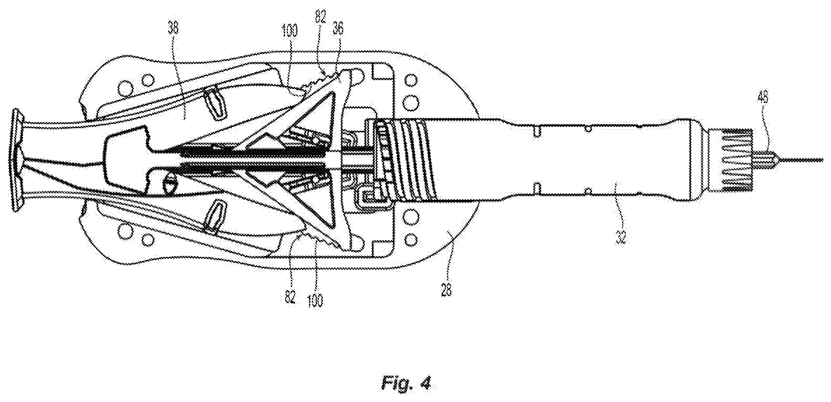

When plunger 38 is at least partially retracted from housing assembly 22, the biasing force exerted by material bridge 108 will tend to draw plunger 38 back into housing assembly 22. FIG. 4 shows plunger 38 in a partially retracted position while FIG. 3 shows plunger 38 in a fully retracted position. When plunger 38 is fully retracted by the user when preparing for an injection event, it is desirable for plunger 38 to remain in the fully retracted position (FIG. 3), until after the user has inserted the needle into the injection site and is ready to begin the injection. Detents 78 on arms 60 engage with each other to retain arms 60 in the fully retracted position.

Advantageously, in the exemplary embodiment, the force required to disengage detents 78 is only slightly greater than the biasing force generated by material bridge 108 so that the user is not required to exert a significant force to separate detents 78. As the user depresses plunger 38 to cause the injection, the biasing force generated by material bridge 108 will assist the user.

Use of a material bridge allows plunger 38 to be formed out of a single molded part instead of two or more parts. The use of a single molded part reduces parts inventory and the assembly labor required to manufacture device 20 thereby enhancing manufacturing efficiency.

Alternative embodiments of a molded one part plunger could employ a button end where the material bridge of the button end is stressed beyond the yield point without fracturing the material whereby the material bridge would not exert a biasing force on arms 60. In such an embodiment, it would be appropriate to describe the material bridge as a living hinge.

Returning to a discussion of the priming of cartridge 24, it is noted that FIGS. 9, 10 and 11 show device 20 in three sequential stages of the priming process. FIGS. 9 and 9A-9C show device 20 with a fresh cartridge 24 disposed within cartridge retainer 32 and with grooves 70 on cartridge retainer 32 engaged with threads 66 on housing assembly 22 sufficiently to hold cartridge retainer 32 on housing assembly 22 with ratchet members 68 having passed over one of the ratchet teeth 72 (FIG. 9B). Threads 66 have been engaged with grooves 70 but have not yet reached the end of grooves 70 as can be seen in FIG. 9C. Because at least one of the ratchet members 68 has passed over at least one of the teeth 72, cartridge retainer 32 cannot be removed without detaching housing members 28, 30 from each other. In the illustrated embodiment, each of the ratchet members 68 pass over an initial tooth 72 at the same time. In this initial configuration, drive stem 34 is pulled rearwardly to the limit of its rearward travel defined by the engagement of ratchet members 95 with the ratchet tooth 88 on stem 34 that is located nearest stem foot 54. As can be seen in FIG. 9A, there is a gap between foot 54 and piston 42 when device 20 is in this configuration. Device 20 may advantageously be packaged and shipped in the condition shown in FIGS. 9, 9A-9C.

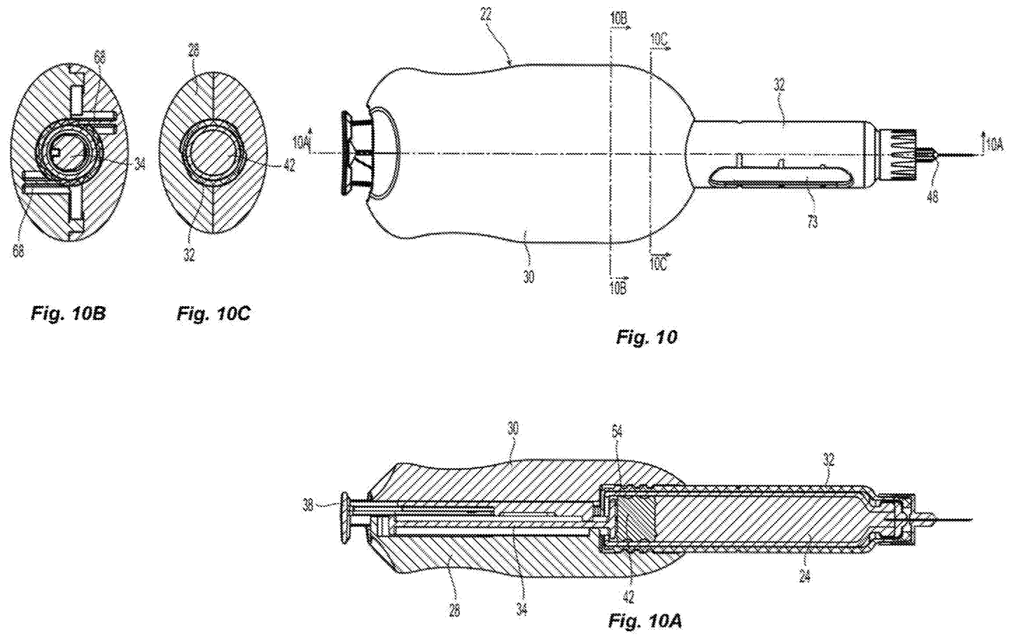

Prior to the initial injection from cartridge 24, the user will prime the cartridge. This will include mounting needle assembly 48 onto threaded end 52 of cartridge retainer 32 and then further rotating cartridge retainer 32 relative to housing assembly 22. FIGS. 10, 10A-10C illustrate device 20 after needle assembly 48 has been installed and after the user has begun to rotate cartridge retainer 32 relative to housing assembly 22 but has not yet completed the rotational movement of cartridge retainer 32. In this partially installed position, the threading engagement of cartridge retainer 32 with housing assembly 22, and more specifically the threading engagement of grooves 70 with threads 66, has drawn cartridge retainer 32 and cartridge 24 into the housing assembly and thereby drawn piston 42 and drive stem foot 54 closer together. As can be seen in FIG. 10A, drive stem foot 54 and piston 42 have not yet made contact but are positioned close to each other. As can be seen in FIG. 10, window 73 faces to the side of device 20. The engagement of ratchet members 68 with ratchet teeth 72 is illustrated in FIG. 10B. Threads 66 have not yet reached the end of grooves 70 and the gap therebetween is illustrated in FIG. 10C.

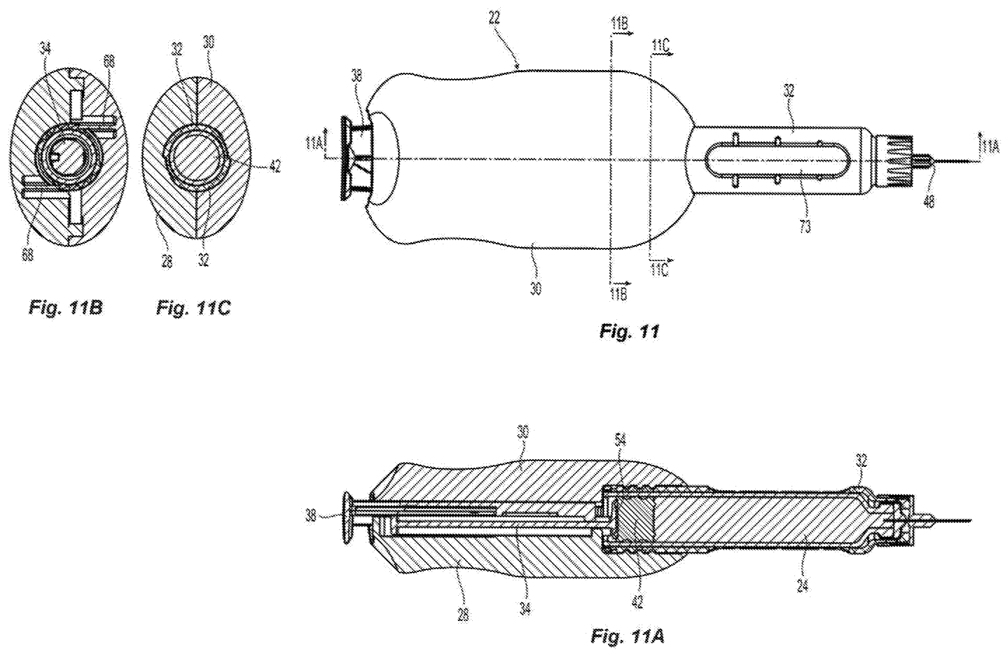

The configuration of device 20 at the completion of the priming procedure is shown in FIGS. 11, 11A-11C. Cartridge retainer 32 has been rotated to engage threads 66 with the ends of grooves 70 such that further rotation of cartridge retainer 32 is not possible as illustrated in FIG. 11C. As shown in FIG. 11B, ratchet members 68 are engaged with ratchet teeth 72 and prevent the rotational withdrawal of cartridge retainer 32 and require that housing members 28, 30 be detached to remove cartridge retainer 32 from housing assembly 22. Windows 73 are positioned on cartridge retainer 32 such that when retainer 32 is in this installed position, windows 73 face in a direction that is perpendicular to the parting line between housing members 28, 30 as illustrated in FIG. 11. As illustrated in FIG. 11A, foot 54 of drive stem 34 is in contact with piston 42 and has slightly advanced piston 42 within cartridge barrel 40 to thereby prime cartridge 24 in preparation for the initial discharge of medication during an injection.

It is noted that the illustrated device 20 has helical grooves 70 and helically extending ratchet teeth 72 that have a length adapted for use with cartridge 24. The length of grooves 70 and ratchet teeth 72 may be adjusted to account for differences in the travel distance required to bring drive stem 34 into contact with piston 42 and prime the cartridge.

It is also noted that the use of a housing assembly and cartridge retainer as exemplified herein to prime a medicament cartridge may be used with a wide variety of different driving mechanisms employing an axially moveable drive element. The priming mechanism disclosed herein may be employed with any number of different injection devices including a housing and cartridge retainer that holds and moves the cartridge as it is moved relative to housing.

It is further noted that the illustrated device 20 is designed to provide a single priming procedure prior to the first injection from cartridge 24. For many medications and applications a single priming procedure for a cartridge 24 will be sufficient. This may be because the cartridge is a single-use cartridge which will be depleted after a single injection or because subsequent injections from same cartridge do not require separate priming events.

For some applications, however, it may be desirable to conduct multiple priming procedures for an individual cartridge. Modification of device 20 may readily provide for such multiple priming procedures. For example, the length of grooves 70 and ratcheting teeth 72 may be extended.

The initial priming event would not involve rotating cartridge retainer 32 until threads 66 reached the end of grooves 70. Instead, the user would rotate cartridge retainer 32 a predefined number clicks, caused by ratchet members 68 passing over an individual tooth 72, for each priming procedure. Alternatively, and/or additionally, in one embodiment housing assembly 22 is provided with a series of marks adjacent cylindrical opening 64 on the exterior surface of housing assembly 22 and cartridge retainer 32 is provided with a pointer or other symbol that is positioned adjacent housing assembly 22 and which moves from one of the marks adjacent cylindrical opening 64 to the next mark as cartridge retainer 32 is threaded into engagement with housing assembly 22. The circumferential distance between the marks on housing 22 defines the amount of rotation the user should employ for an individual priming procedure, such that, when priming the device, the user would twist cartridge retainer 32 until the pointer on cartridge retainer 32 was aligned with the next sequential mark located on housing member 22. Various other modifications may also be employed with the device of the present application.

Device 20 may further comprise a drug or medication. In one embodiment, a system may comprise one or more devices including device 20 and a drug. The term drug or medication refers to one or more therapeutic agents including but not limited to insulins, insulin analogs such as insulin lispro or insulin glargine, insulin derivatives, glucagon-like peptide (GLP-1) receptor agonists such as dulaglutide or liraglutide, glucagon, glucagon analogs, glucagon derivatives, gastric inhibitory polypeptide (GIP), GIP analogs, GIP derivatives, oxyntomodulin analogs, oxyntomodulin derivatives, therapeutic antibodies and any therapeutic agent that is capable of delivery by device 20. The drug as used in device 20 may be formulated with one or more excipients. Device 20 is operated in a manner generally as described above by a patient, caregiver or healthcare professional to deliver drug to a person.

While this invention has been described as having exemplary designs, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An injection device for dispensing medication from a medicament cartridge having a needle assembly at one end and a piston disposed opposite the needle assembly wherein advancement of the piston within the cartridge discharges medication from the needle assembly, the injection device comprising:
a housing assembly;
a driving assembly supported in the housing assembly and adapted to drivingly engage the piston to thereby discharge medication from the needle assembly;
a cartridge retainer securable to the housing assembly and adapted to retain the cartridge in a position wherein the driving assembly is operably engageable with the piston of the cartridge; and
wherein securing the cartridge retainer to the housing assembly with a retained cartridge moves the piston of the cartridge into contact with the driving assembly and performs a priming procedure.

2. The injection device of claim 1 wherein the cartridge retainer is securable to the housing assembly by rotationally engaging a helical thread disposed on one of the cartridge retainer and housing assembly with a helical groove disposed on the other one of the cartridge retainer and housing assembly.

3. The injection device of claim 2 wherein one of the cartridge retainer and the housing assembly defines a plurality of ratchet teeth and the other one of the cartridge retainer and the housing assembly defines a ratchet member engagable with the ratchet teeth wherein rotational engagement of the helical thread with the helical groove engages the ratchet member with the ratchet teeth and prevents rotational disengagement of the helical thread with the helical groove.

4. The injection device of claim 2 wherein actuation of the driving assembly linearly translates a drive stem along an axis to drive the piston and wherein the axis defines the rotational axis of the cartridge retainer as the cartridge retainer is being rotationally engaged with the housing assembly.

5. The injection device of claim 1 wherein the device is adapted to perform a priming procedure by relative movement between the cartridge retainer and the housing assembly and is adapted to perform a discharge for an injection by relative movement between the driving assembly and the housing assembly.

6. The injection device of claim 5 wherein the relative movement between the cartridge retainer and the housing assembly includes rotation of the cartridge retainer relative to the housing assembly and wherein the driving assembly includes a drive stem and the relative movement between the driving assembly and the housing assembly includes linear translation of the drive stem.

7. The injection device of claim 1 wherein:
the housing assembly defines a pair of ramps;
the driving assembly includes a driver operably coupled with the piston and adapted to advance the piston within the cartridge;
a plunger having a pair of arms, the plunger being moveable between a retracted position and an advanced position by a force exerted on the plunger, each of the arms being engageable with a respective one of the ramps whereby when the plunger is moved from the retracted position to the advanced position, the movement of the arms is guided by the ramps and the plunger moves the driver to thereby advance the piston.

8. The injection device of claim 7 wherein the cartridge retainer is securable to the housing assembly by rotationally engaging a helical thread disposed on one of the cartridge retainer and housing assembly with a helical groove disposed on the other one of the cartridge retainer and housing assembly.

9. The injection device of claim 8 wherein one of the cartridge retainer and the housing assembly defines a plurality of ratchet teeth and the other one of the cartridge retainer and the housing assembly defines a ratchet member engagable with the ratchet teeth wherein rotational engagement of the helical thread with the helical groove engages the ratchet member with the ratchet teeth and prevents rotational disengagement of the helical thread with the helical groove.

10. The injection device of claim 8 wherein the driving assembly further includes a drive stem engageable with the piston and wherein actuation of the driving assembly engages the driver with the drive stem to linearly translate the drive stem along an axis to thereby drive the piston and wherein the axis defines the rotational axis of the cartridge retainer as the cartridge retainer is being rotationally engaged with the housing assembly.

11. The injection device of claim 7 wherein the device is adapted to perform a priming procedure by relative movement between the cartridge retainer and the housing assembly and is adapted to perform a discharge for an injection by relative movement between the driving assembly and the housing assembly.

12. The injection device of claim 11 wherein the relative movement between the cartridge retainer and the housing assembly includes rotation of the cartridge retainer relative to the housing assembly and wherein the driving assembly includes a drive stem operably disposed between the driver and the piston and the relative movement between the driving assembly and the housing assembly includes linear translation of the drive stem.

13. The injection device of claim 1, further including the medication contained in the cartridge.
